(19) United States
(12) Patent Application Publication
(10) Pub. No.: US 2023/0048008 A1
(43) Pub. Date: Feb. 16, 2023

(54) STABILIZATION OF OXIDASES BY GLYCINE

(71) Applicant: DANISCO US INC, Palo Alto, CA (US)

(72) Inventors: ANGELA CIFELLI, PALO ALTO, CA (US); PEYMAN MOSLEMY, PALO ALTO, CA (US); NGUYEN PHAM, PALO ALTO, CA (US)

(21) Appl. No.: 17/786,726

(22) PCT Filed: Dec. 18, 2020
(86) PCT No.: PCT/US2020/066068

§ 371 Date: Jun. 17, 2022

Related U.S. Application Data
(60) Provisional application No. 62/949,542, filed on Dec. 18, 2019.

(51) Int. Cl. C12N 9/96; C12N 9/04

(52) U.S. Cl. CPC C12N 9/96; C12N 9/0006; C12Y 101/03004; C12Y 101/03005

(57) ABSTRACT

Described are compositions and methods relating to the stabilization of oxidase enzymes using the amino acid glycine. The compositions and methods allow for drying oxidase-containing compositions with improved enzyme activity yield and long term storage of dried oxidase-containing compositions, in some cases without the need for refrigeration.

### CROSS REFERENCE

This application is a 371 of International Application No. PCT/US2020/066068 filed Dec. 18, 2020, which claims the benefit of U.S. Patent Application No. 62/949542, filed Dec. 18, 2019, all of which are incorporated by reference in their entirety.

### FIELD OF THE INVENTION

The present compositions and methods relate to the stabilization of oxidase enzymes using the amino acid glycine. The compositions and methods allow for heat drying oxidase-containing compositions with improved enzyme activity yield and long term storage of dried oxidase-containing compositions, in some cases without the need for refrigeration.

### BACKGROUND

Oxidases (EC 1.1.3) are enzymes that catalyze oxidation-reduction reactions, typically involving diatomic oxygen (O₂) as an electron acceptor. Examples of oxidases are hexose oxidase, glucose oxidase, monoamine oxidase, xanthine oxidase, L-gulonolactone oxidase, lysyl oxidase, NADPH oxidase, polyphenol oxidase, cytochrome P450 oxidase and laccase. Numerous additional enzymes are listed, herein.

Glucose oxidase (GOx; EC 1.1.3.4) is an oxidase that can be over-expressed in heterologous hosts for large-scale production and has a particularly broad range of commercial uses. GOx, sometimes referred to as GOD, is widely used to control microbial contamination, e.g., in wine making, and in biochemical assays and biosensors to measure free glucose, e.g., in blood and urine. GOx is also used to produce stronger dough in baking, to remove oxygen in food packages, and to prevent the browning of certain foods, such as egg whites.

GOx is a thermally sensitive enzyme and commercial production is made expensive and inefficient by considerable activity loss during production and storage. Processes such as a spray drying and pelleting to make animal feed are particularly destructive to GOx. In view of the myriad uses of GOx and other oxidases, the need exists for ways to increase stabilization in a cost-effective manner.

Hexose oxidase (HOx: EC 1.1.3.5) catalyzes the oxidation of mono- and disaccharides to their corresponding lactones, with concomitant reduction of molecular oxygen to hydrogen peroxide. This enzyme is produced commercially by over-expression in certain methylotrophic yeasts. Hexose oxidase is able to oxidize a variety of substrates including D-glucose, D-galactose, maltose, cellobiose, and lactose. The wide substrate specificity distinguishes this enzyme from GOx which is highly specific for D-glucose. HOx is also used to produce stronger dough in baking.

GOx and HOx are sensitive enzymes and their commercial production is made expensive and inefficient by considerable activity loss during production and storage. The need exists for ways to increase stabilization in a cost-effective manner.

### SUMMARY

The present compositions and methods relate to the stabilization of oxidases using the amino acid glycine. The compositions and methods allow for heat drying oxidase-containing compositions with improved enzyme activity yield and long term storage of dried oxidase-containing compositions with reduced the need for refrigeration. Aspects and embodiments of the compositions and methods are described in the following, independently-numbered, paragraphs.

1. In one aspect, a method for increasing the stability of an oxidase enzyme in a composition is provided, comprising; admixing with the oxidase enzyme the free amino acid glycine at a ratio of at least 1 gram glycine per gram oxidase enzyme, wherein the admixed oxidase enzyme has increased stability in the composition compared to an oxidase enzyme in an otherwise identical composition lacking glycine.

2. In some embodiments of the method of paragraph 1, the ratio is at least 6.4 gram glycine per gram oxidase enzyme.

3. In some embodiments of the method of paragraph 1 or 2, glycine is the primary stabilizer of oxidase enzyme in the formulation.

4. In some embodiments of the method of any previous paragraphs, glycine is present in the substantial absence of free acidic amino acids.

5. In some embodiments of the method of any previous paragraphs, glycine is the only free amino acid present.

6. In some embodiments of the method of any previous paragraphs, the oxidase enzyme is glucose oxidase or hexose oxidase.

7. In some embodiments of the method of any of the previous paragraphs, the oxidase enzyme and glycine are admixed in an aqueous solution or suspension.

8. In some embodiments of the method of paragraph 7, the admixed oxidase enzyme and glycine are subsequently dried.

9. In another aspect, a composition comprising an oxidase enzyme and the free amino acid glycine at a ratio of at least 1 gram glycine per gram oxidase enzyme is provided, wherein the oxidase enzyme has increased stability in the composition compared to an oxidase enzyme in an otherwise identical composition lacking glycine.

10. In some embodiments of the composition of paragraph 9, the ratio is at least 6.4 gram glycine per gram oxidase enzyme.

11. In some embodiments of the composition of paragraph 9 or 10, glycine is present in the substantial absence of acidic amino acid residues.

12. In some embodiments of the composition of any of paragraphs 9-11, glycine is the only free amino acid present.

13. In some embodiments of the composition of any of paragraphs 9-12, the oxidase enzyme is glucose oxidase hexose oxidase.

14. In some embodiments of the method or composition of any of paragraphs 9-13, the oxidase enzyme and glycine are incorporated into a granule, a film, a pad, a gel, or other solid and/or liquid composition.

These and other aspects and embodiments of the compositions and methods will be apparent from the present description.

### BRIEF DESCIPTION OF THE DRAWING

FIG. 1 is a graph showing the the ratio of bound to free FAD in the presence of increasing amounts of glycine per gram active GOx.

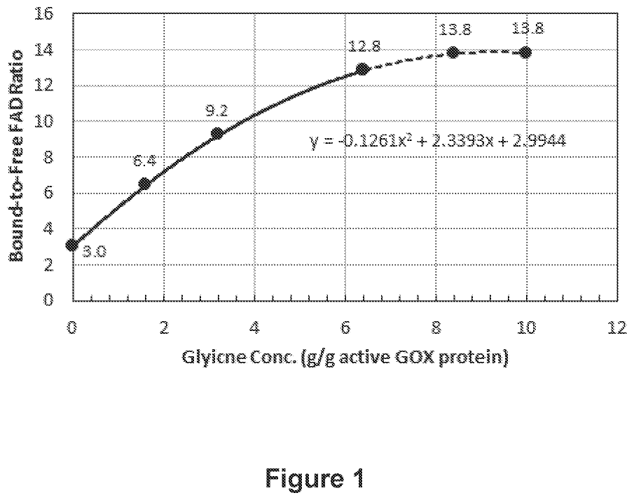

### DETAILED DESCRIPTION OF THE INVENTION

### I. Introduction

The inventors have discovered that incorporation of the amino acid glycine, more than any other amino acid, improves the heat drying yield in production of enzyme compositions from formulated oxidase enzyme concentrates. While stabilization of oxidases, such as glucose oxidase (GOx), using amino acids has been described (see, e.g., U.S. Pat. No. 4,543,326), such studies have consistently identified different amino acid residues, such as acidic amino acid residues (i.e., aspartic acid and glutamic acid, and salts, thereof) as being the most preferable amino acids for stabilization. The present selection invention relates to the use of glycine, a neutral amino acid, specifically, as a preferred stabilizer of oxidases.

### II. Definitions and abbreviations

Prior to describing the present compositions and methods in detail, the following terms are defined for clarity. Terms not defined should be accorded their ordinary meanings as used in the relevant art.

As used herein, the term “granule” refers to a small particle of a substance. The particle comprises a core, optionally with one or more coating layers.

As used herein, “weight percent,” “weight fraction,” “mass fraction” or simply “fraction” refers to the relative amount of mass on a % wt/wt or fractional wt/wt basis, for example, the relative amount of mass of an ingredient compared to the mass of an entire granule.

As used herein, the terms “pellets” and “pelleting” refer to solid, rounded, spherical and cylindrical tablets or pellets and the processes for forming such solid shapes, particularly feed pellets and solid, extruded animal feed.

As used herein, the term “recovered activity” or “activity recovery” refers to the ratio of (i) the activity of an enzyme after a treatment involving one or more of the following stressors: heating, increased pressure, increased pH, decreased pH, storage, drying, exposure to surfactant(s), exposure to solvent(s), and mechanical stress) to (ii) the activity of the enzyme before the treatment. The recovered activity may be expressed as a percentage. The percent recovered activity is calculated as follows:

$$\% recovered activity = \frac{( activity after treatment )}{( activity before treatment )} \times 100 \%$$

As used herein, the term “about” refers to ±15% to the referenced value.

For ease of reference, elements of the present compositions and methods may be arranged under one or more headings. It is to be noted that the compositions and methods under each of the headings also apply to the compositions and methods under the other headings.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:

° C. degrees Centigrade

g or gm gram

g/L grams per liter

g/mol grams per mole

mol/mol mole to mole ratio

μmol micromole

hr or h hour

kg kilogram

mg milligram

mL or ml milliliter

min minute

M molar

mM millimolar

μm micrometer (micron)

μL and μl microliter

UFC ultrafiltered concentrate

dissolved solids active oxidase protein plus other non-oxidase fermentation solids

RH relative humidity

wt weight

% wt/wt weight percent

### III. Oxidases that can be stabilized by glycine

The discovery that glycine is the preferred amino acid stabilizer for exemplified oxidase enzymes, is expected to apply to a broad range of oxidases. Moreover, testing glycine for the ability to stabilize a particular oxidase is routine and does not require undue experimentation.

Particular oxidases expected to be stabilized using glycine are those that use molecular oxygen (02) as an acceptor, and are classified as EC 1.1.3. Exemplary oxidases include those listed in Table 1.

TABLE 1
EC 1.1.3 enzymes

| Enzyme classification | Common name |
|---|---|
| EC 1.1.3.3 | malate oxidase |
| EC 1.1.3.4 | glucose oxidase |
| EC 1.1.3.5 | hexose oxidase |
| EC 1.1.3.6 | cholesterol oxidase |
| EC 1.1.3.7 | aryl-alcohol oxidase |
| EC 1.1.3.8 | L-gulonolactone oxidase |
| EC 1.1.3.9 | galactose oxidase |
| EC 1.1.3.10 | pyranose oxidase |
| EC 1.1.3.11 | L-sorbose oxidase |
| EC 1.1.3.12 | pyridoxine 4-oxidase |
| EC 1.1.3.13 | alcohol oxidase |
| EC 1.1.3.14 | catechol oxidase (dimerizing) |
| EC 1.1.3.15 | (S)-2-hydroxy-acid oxidase |
| EC 1.1.3.16 | ecdysone oxidase |
| EC 1.1.3.17 | choline oxidase |
| EC 1.1.3.18 | secondary-alcohol oxidase |
| EC 1.1.3.19 | 4-hydroxymandelate oxidase |
| EC 1.1.3.20 | long-chain-alcohol oxidase |
| EC 1.1.3.21 | glycerol-3-phosphate oxidase |
| EC 1.1.3.23 | thiamine oxidase |
| EC 1.1.3.24 | L-galactonolactone oxidase |
| EC 1.1.3.27 | hydroxyphytanate oxidase |
| EC 1.1.3.28 | nucleoside oxidase |
| EC 1.1.3.29 | N-acylhexosamine oxidase |
| EC 1.1.3.30 | polyvinyl-alcohol oxidase |
| EC 1.1.3.37 | D-arabinono-1,4-lactone oxidase |
| EC 1.1.3.38 | vanillyl-alcohol oxidase |
| EC 1.1.3.39 | nucleoside oxidase (H2O2-forming) |
| EC 1.1.3.40 | D-mannitol oxidase |
| EC 1.1.3.41 | xylitol oxidase |
| EC 1.1.3.42 | prosolanapyrone-II oxidase |
| EC 1.1.3.43 | paromamine 6′-oxidase |
| EC 1.1.3.44 | 6-hydroxyneomycin C oxidase |

The oxidase enzyme is preferably stabilized with glycine in an aqueous mixture but may subsequently be dried, such as by spray drying, spray agglomeration, spray granulation etc. The oxidase enzyme may be subjected to high shear and extrusion followed by drying, and may used in fluid bed coating. The glycine stabilized oxidase may be incorporated into a granule,a pellet, a film, a pad, a gel, or any other solid or liquid composition, preferably one that does not separate glycine from the oxidase.

### IV. Ratio of glycine to oxidase enzyme

It is likely that different oxidases will require different amounts of glycine for stabilization, which amounts are readily determined. Such amounts are best expressed as the molar ratio or weight percentage of glycine to active oxidase enzyme protein or, alternatively, glycine to oxidase enzyme activity (in defined units). Based on the appended Examples, the recommended ratio is about 1 to about 10 g glycine per g active oxidase protein, for example about 1.6 to about 8.4 g glycine per g active oxidase protein. In some case, the ratio is at least about 6.4 g glycine per g active oxidase protein.

The origin of the glycine, i.e., whether naturally occurring or synthetic, is not critical. It will be appreciated the glycine used as a stabilizer is free glycine and not glycine residues incorporated into a protein, including the oxidase protein to be stabilized. Glycine is the primary determinant for oxidase stabilization, meaning it is the only excipient necessary and sufficient for improved stability; however, glycine may also be mixed with other amino acids, other stabilizing agents, or other beneficial agents.

### EXAMPLES

The following examples are intended to be illustratative, but not limit the scope of this invention to the exemplified oxidases. The glucose oxidase (GOx) enzyme also known as Notatin (EC number 1.1.3.4) is an oxidase that catalyzes the oxidation of glucose to hydrogen peroxide and D-glucono-δ-lactone. This enzyme is produced by certain species of fungi such as Aspergillus niger. The oxidation reaction is performed by flavin adenine dinucleotide (FAD), a redox cofactor which is tightly bound, not covalently, deep between two identical GOx monomers. The GOx dimer contains two FAD cofactors that are responsible for the oxidation-reduction properties of the enzyme. GOx is a thermally labile enzyme and is sensitive to drying processes that involve heating. Under denaturing conditions, such as those occurring during heat drying, the dimer is dissociated to its subunits, leading to irreversible loss of cofactors, transitioning from the “bound” to “free” state, which in turn leads to enzyme inactivation and aggregation.

### Example 1. Thermal inactivation of GOx during heat drying

Two samples of neat (unformulated) GOx UFC (25 mg active protein per g UFC) were obtained from DuPont/Danisco fermentation plants. The samples were incubated in an oven at 50° C. for four, eight and twenty-four hours. The experimental method involved addition of 80 μL of each sample to interior wells of a 96-well plate (to avoid edge effects), sealing the plate with breathable seal, and incubaring the plate at 50° C. The weight of the plate was recorded before and after drying to ensure all water was removed.

To measure activity, the dried content of each well was re-suspended in 80 μL of purified water. After resuspension, the activity was measured using a standard glucose oxidase assay. Glucose oxidase catalyzes the conversion of glucose and oxygen to hydrogen peroxide and gluconic acid in the assay. A reaction of hydrogen peroxide with 2,2′-azino-bis(3-ethylbenzthiazolin-6-sulfonsyre), ABTS, changes the appearance of reaction media from colorless to green. This reaction is catalyzed by the enzyme peroxidase. The green color is measured on a spectrophotometer at 405 nm. The method is calibrated using a linear regression of standard dilutions prepared from a standard material with a known concentration. The oxidase enzymatic activity decreased with time as shown in Table 2.

TABLE 2
The oxidase activity yields of neat GOx UFC samples incubated at 50°
C. for 4, 8, and 24 hours; n = 8, avg. ± stdev

| | % Recovered Activity | | |
|---|---|---|---|
| Unformulated GOx UFC | 4 hr | 8 hr | 24 hr |
| Sample #1 | 81% ± 2.8% | 52% ± 3.4% | 31% ± 3.6% |
| Sample #2 | 71% ± 4.8% | 34% ± 7.4% | 13% ± 11% |

### Example 2. Thermal stabilization of GOx by glycine

A sample of neat (unformulated) GOx UFC (25 mg active protein per g UFC) was obtained from a DuPont/Danisco fermentation plant. A series of thermal stress experiments was conducted on the GOx concentrate formulated with different concentrations of glycine ranging from 0%-16%. A correlation was observed between GOx denaturation (based on the ratio of the bound to free FAD cofactors) and glycine concentration after heating the samples for 8 hours at 50° C. (Table 3). FAD profiling of GOx samples was performed by hydrophobic interaction chromatography with UV detection at 450 nm. The area under the absorption curve vs. retention time was determined for “bound” and “free” FAD cofactors, and their respective ratio was then calculated. Addition of glycine to the GOx UFC reduced the degree of denaturation of enzyme protein under the heat stress. Based on graphical analysis of the denaturation profile, preferred glycine concentrations are greater than about 1 g glycine per g oxidase protein, for example greater than 1.6, or even greater than 6.4 g glycine per g oxidase protein. Amounts greater than about 8.4 g glycine per g oxidase protein do not apper to provide much additional benefit.

TABLE 3
Variation of free and bound FAD with glycine concetration

| % Glycine to | Glycine to | | | |
|---|---|---|---|---|
| Gox Conc. | Gox Mass Ratio | | | Bound |
| Ratio | (g glycine/ | | | to Free |
| (w glycine/ | g active | Free | Bound | FAD |
| w UFC) | protein) | FAD | FAD | Ratio |
| 0% | 0 | 184.7 | 545.9 | 3.0 |
| 4% | 1.6 | 126.7 | 805.7 | 6.4 |
| 8% | 3.2 | 83.2 | 761.5 | 9.2 |
| 16% | 6.4 | 61.3 | 784.4 | 12.8 |

Samples of neat GOx UFC and formulated GOx UFC containing a cationic or neutral amino acid were prepared for drying in a 96-well plate. About 20 μL of each GOx preparation was added to 6 wells and the plate was sealed with a breathable cover. The plate was incubated at 50° C. for 4 hours to dry the samples. Enzyme activity of dried samples was measured by the protocol descrbed in Example 1 above.

Cationic amino acids included arginine and lysine, and neutral amino acids included alanine, glycine, proline, and threonine. Among the amino acids tested, alanine, glycine and proline showed the highest solubility in the GOx UFC. Histidine, a neutral amino acid, was not included, despite its high solubility, due to its tendency to increase the pH of the concentrate.

The recovered activity of GOx formulated with different cationic and neutral amino acids is shown in Table 4. Anionic amino acids including aspartic acid, D-glutamic acid, and L-glutamic acid had considerably lower solubility in the GOx UFC, and showed inferior enzyme activity yield compared to neutral and cationic amino acids. As such, they are not preferred excipients for thermal stabilization of GOx compositions of this invention.

TABLE 4
Recovered activity of GOx after drying at 50° C.
for 4 hours; n = 6, avg ± stdev

| | | AA to GOx | AA to GOx | |
|---|---|---|---|---|
| | AA side | mass ratio | mass ratio | |
| Amino | chain | (g AA/ | (mM AA/ | Recovered |
| acid | charge at | g active | g active | activity |
| (AA) | pH 7.4 | protein) | protein) | (%) |
| — | — | 0 | 0 | 73.87% ± 5.43% |
| Arginine | positive | 3.8 | 21.81 | 73.73% ± 6.63% |
| Lysine | positive | 3.7 | 25.31 | 93.63% ± 7.70% |
| Alanine | neutral | 4.0 | 44.90 | 88.74% ± 6.46% |
| Glycine | neutral | 4.0 | 53.29 | 102.38% ± 6.80% |
| Proline | neutral | 4.1 | 35.61 | 88.44% ± 8.76% |
| Theronine | neutral | 3.9 | 32.74 | 91.35% ± 8.59% |

The activity assay results showed that the dried formulation with glycine had the highest recovered activity compared to the neat sample without glycine and the samples formulated with other neutral and cationic amino acids. Glycine has the lowest molecular weight among other amino acids tested, enabling its incorporation as a thermal stabilizer at the highest molar concentration relative to active enzyme protein.

### Example 3. Storage stability of GOx spray dried compositions

Addition of glycine to GOx concentrate, UFC dissolved solids 8.2%, at 14% (wt glycine/wt UFC), i.e. 5.6 g glycine per g active protein, improved the storage stability of dried enzyme composition produced with spray drying. The spray dried GOx powder compositions with glycine and without glycine (control) were subjected to a 6-month stability study. The samples were contained in closed cap plastic bottles and incubated at 37° C. and 65% relative humidity for 180 days. The enzyme activity was monitored during this period by analyzing the samples after 0, 3, 7, 14, 30, 60, 90 and 180 days. The recovered enzymatic activity of the tested samples is tabulated in Table 5. The GOx/glycine composition maintained up to 55% of enzyme activity after 180 days in storage.

TABLE 5
Recovered enzymatic activity (%) of glucose oxidase/glycine
spray dried powder composition in comparison with the unformulated
glucose oxidase after 6-month storage at 37° C./65% RH

| | Time (days) | | | | | | | |
|---|---|---|---|---|---|---|---|---|
| | 0 | 3 | 7 | 14 | 30 | 60 | 90 | 180 |
| Samples | Recovered activity (%) | | | | | | | |
| Unformulated | 100 | 86 | 86 | 75 | 74 | 61 | 50 | 28 |
| GOx/glycine | 100 | 90 | 88 | 80 | 81 | 69 | 60 | 55 |

What is claimed is:

1. A method for increasing the stability of an oxidase enzyme in a composition, comprising; admixing with the oxidase enzyme the free amino acid glycine at a ratio of at least 1 gram glycine per gram oxidase enzyme, wherein the admixed oxidase enzyme has increased stability in the composition compared to an oxidase enzyme in an otherwise identical composition lacking glycine.

2. The method of claim 1, wherein the ratio is at least 6.4 gram glycine per gram oxidase enzyme.

3. The method of claim 1 or 2, wherein glycine is the primary stabilizer of oxidase enzyme in the formulation.

4. The method of any previous claims, wherein glycine is present in the substantial absence of free acidic amino acids.

5. The method of any previous claims, wherein glycine is the only free amino acid present.

6. The method of any previous claims, wherein the oxidase enzyme is glucose oxidase or hexose oxidase.

7. The method of any of the previous claims, wherein the oxidase enzyme and glycine are admixed in an aqueous solution or suspension.

8. The method of claim 7, wherein the admixed oxidase enzyme and glycine are subsequently dried.

9. A composition comprising an oxidase enzyme and the free amino acid glycine at a ratio of at least 1 gram glycine per gram oxidase enzyme, wherein the oxidase enzyme has increased stability in the composition compared to an oxidase enzyme in an otherwise identical composition lacking glycine.

10. The composition of claim 9, wherein the ratio is at least 6.4 gram glycine per gram oxidase enzyme.

11. The composition of claim 9 or 10, wherein glycine is present in the substantial absence of acidic amino acid residues.

12. The composition of any of claims 9-11, wherein glycine is the only free amino acid present.

13. The composition of any of claims 9-12, wherein the oxidase enzyme is glucose oxidase hexose oxidase.

14. The method or composition of any of claims 9-13, wherein the oxidase enzyme and glycine are incorporated into a granule, a film, a pad, a gel, or other solid and/or liquid composition.
